(19) United States
(12) Patent Application Publication
(10) Pub. No.: US 2025/0144323 A1
(43) Pub. Date: May 8, 2025

(54) Insufflation Apparatus with Novel Pressure Measurement

(71) Applicant: W.O.M. World of Medicine GmbH, Berlin (DE)

(72) Inventors: Henrik Stier, Berlin (DE); Jan-Hendrik Carstens, Berlin (DE)

(73) Assignee: W.O.M. World of Medicine GmbH, Berlin (DE)

(21) Appl. No.: 18/836,819

(22) PCT Filed: Feb. 8, 2023
(86) PCT No.: PCT/EP2023/053125

§ 371 Date: Aug. 8, 2024

(30) Foreign Application Priority Data
Feb. 8, 2022 (DE) 102022000487.9

(51) Int. Cl. A61M 13/00; A61M 39/24

(52) U.S. Cl. CPC A61M 13/003; A61M 39/24; A61M 2039/242; A61M 2202/0007; A61M 2202/02; A61M 2205/18; A61M 2205/3327; A61M 2205/3334; A61M 2205/3337; A61M 2205/50

(57) ABSTRACT

The present invention relates to an insufflator with a novel pressure measuring device for minimally invasive surgery. The new device allows to reliably measure the pressure in the body cavity while at the same time ensuring that the gas supply lines are free of occlusions.

The present invention relates to an insufflator with a novel pressure measuring device. The new device allows to reliably measure the pressure in the body cavity while at the same time ensuring that the gas supply lines are free.

### BACKGROUND AND PRIOR ART

Various designs of insufflators are known from prior art. Insufflators typically have a tube through which a medical gas is introduced into a body cavity (e.g., an abdomen). The gas creates a positive pressure, which expands the body cavity so that there is enough space for visual inspection or therapeutic intervention. Optional embodiments allow the gas to be sucked out of the abdominal cavity via a second tube. Embodiments of this type are used in particular in therapeutic interventions by means of electrosurgery or lasers in order to remove and filter harmful smoke gases.

In practice, it has proven difficult to accurately determine the pressure in the body cavity. Various configurations of measuring devices are also known for determining pressure in the body cavity. The possibility of measuring the pressure by means of a pressure sensor in the body cavity itself has the disadvantage that, in addition to the necessary medical instruments, the pressure sensor also has to be positioned in the body cavity. On the one hand, this means an additional element in the body cavity, with associated connections, which takes up space and at the same time represents a risk of infection; on the other hand, this results in higher costs for the pressure sensor, because it either has to be cleaned and sterilised in a complex manner or has to be designed as a disposable article.

Embodiments have therefore become established in which the pressure sensor is not located in the body cavity itself, but rather in one of the tubes, more or less spaced apart from the body cavity. The inherent disadvantage of this sensor arrangement is that there is a pressure difference between the body cavity and the measuring position (e.g., in the tube), caused by the tube, the trocar and possibly other devices. It is not easy to determine this pressure loss and this is the subject of many investigations. The farther away the pressure sensor is from the body cavity, the greater the deviations of the measured pressure from the pressure in the body cavity. The closer the pressure sensor is to the gas supply apparatus (e.g., valve-controlled pump) or to a suction pump, the greater the deviations caused by the activity of the pumps (e.g., pressure pulses) or of the valves. Moreover, the effects are superimposed by errors which arise from the fact that the tubes are closed and do not allow fluid flow to or from the body cavity. Such a blockage can be caused by a bent tube or by the fact that the opening in the patient is closed by body tissue. In such a case of the tube occlusion, the pressure sensor only measures the pressure in the tube, not the pressure in the body cavity, which leads to considerable safety concerns.

DE 202004 021703 U1 further discloses a device for delivering substances (e.g., chemotherapeutic agents) into an insufflated body cavity, which, however, does not allow the measurement errors mentioned above to be avoided, in particular no detection of tube occlusion.

In order to overcome the above-mentioned disadvantages of existing pressure measurement systems, the problem has arisen to provide an insufflator which ensures a precise pressure measurement and reliably detects a tube occlusion in case it occurs.

The present invention therefore relates to an insufflator for minimally invasive surgery, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second controllable valve (11) and a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
wherein the sensor line (9) is connected to the first pressure and flow control unit (2), and wherein the second controllable valve (11) allows a pulsatile gas flow controlled by the computing unit (14) into the cavity (8).

Further embodiments of the invention, including the associated operating methods, are described below and are partly the subject matter of accessory and dependent claims.

### Principles of the Invention and Simple Embodiments

The invention is shown in principle in FIG. 1. A gas connection for a gas cylinder or domestic gas can be seen, which leads to a first pressure and flow control unit with a proportional valve. The line then leads to a controllable valve. The first pressure and flow control unit can provide a gas flow of up to 50 litres/min at a pressure of 2 to 3 bar. In addition, a gas flow sensor for measuring the gas flow as well as a first gas pressure sensor can be seen. The supply line leads into the body cavity via the tube and the trocar.

It can also be seen that a sensor line branches off between the first pressure and flow control unit and the first controllable valve. This sensor line has a significantly smaller cross-section (3 to 5 mm) and is configured for a gas flow of less than 1 litre/min at the pressure of 2 to 3 bar mentioned above. The gas flow through the sensor line is preferably less than 0.5 litre/min, most preferably less than 0.1 litres/min. The sensor line may contain a throttle for the purpose of limiting the gas flow; optionally, the throttle may be adjustable or controllable. The sensor line first leads to a second controllable valve and then to a second gas pressure sensor. The sensor line also leads into the body cavity via a second tube and a second trocar. During operation, a discontinuous, pulsatile gas flow via the sensor line can be set via the second controllable valve, as shown in FIG. 1b. For example, a single pressure pulse of 20-50 mmHg can be fed into the sensor line for 1 to 3 seconds, followed by a no-pulse period of 3 to 20 seconds. Due to the comparatively low gas flow through the sensor line, the gas flow through the sensor line practically does not influence the pressure in the cavity. During a gas pulse, on the other hand, the pressure measured by the second gas pressure sensor in the sensor line increases significantly and drops again after closing the second controllable valve until it is identical to the pressure in the body cavity. The actual pressure in the body cavity therefore corresponds to the equilibrium pressure during the no-pulse periods. In case of occlusion of the sensor line, the pressure measured by the second gas pressure sensor increases and remains at the increased pressure level for a longer time (see FIG. 1b). Such a pressure behaviour clearly indicates the occlusion of the sensor line. In this case, an alarm signal is triggered if the increased pressure measured at the second gas pressure sensor lasts for longer than twice the pulse width.

For the detection of an occlusion of the sensor line, therefore, the pulsatile gas flow through the sensor line is decisive in the first place. Without the gas flow pulses, a detection of a sensor line occlusion is not possible in this embodiment of the invention. Furthermore, the comparison of the measurement values of the first gas pressure sensor (5) and the second gas pressure sensor (12) is essential for the function of the described device according to the invention and the insufflation methods carried out therewith.

The triggered alarm signal may be optical and/or acoustic in nature. It is also possible to automatically limit the gas inflow during insufflation in order to avoid excessive pressure in the cavity.

However, the structure of the insufflator according to the invention also allows for a modified mode of operation, which is described below: In this mode of operation, a continuous gas flow through the sensor line is established by keeping the second controllable valve constantly open. With a constant gas flow through the sensor line, a pressure can be read out at the second gas pressure sensor, which is dependent from the pressure in the cavity. However, the pressure measurement at the second gas pressure sensor must be corrected by the pressure loss of the line and the trocar between the second gas pressure sensor and the cavity. For this purpose, the following equation 1 applies:

$${p}_{cavity} = {p}_{sensor} - {p}_{sensor line} \qquad Eq . 1$$

The pressure loss in the sensor line is approximately constant at the mentioned low gas flow through the line and can be determined by measurement or calculation. In this mode of operation, the pressure measured by the second gas pressure sensor increases when an occlusion occurs. The pressure increase by at least 20 mmHg takes place within one to two seconds and drops approximately as quickly when the occlusion is resolved, e.g. in the case of a temporarily bent tube. Such a rapid pressure increase or pressure loss can occur only in the case of an occlusion of the sensor line. The pressure increase in the body cavity in the event of a faulty control of the gas supply line would be significantly slower.

In another embodiment of the invention, a check valve which opens at a low back pressure on the side of the cavity of 8-12 mmHg is included in the sensor line between the second trocar and the second gas pressure sensor. In specific embodiments of the invention, the check valve can open at lower back pressures, for example at 3 mmHg to 5 mmHg, or even at 1 mmHg to 3 mmHg. In this way, it can be checked whether the sensor line is properly connected to the cavity and that there is no occlusion. If there is no connection to the cavity or if an occlusion occurs between the cavity and the second sensor, the check valve remains closed. This situation can be detected by the fact that opening the second controllable valve causes a rapid increase in the measured pressure at the second gas pressure sensor, but not at the first gas pressure sensor (see FIG. 2b). With a proper connection, the measured pressure at the second gas pressure sensor increases a lot more slowly. At the same time, the measured pressure at the first gas pressure sensor increases. In this embodiment, it is also necessary to generate a pulsatile gas flow through the sensor line. Without the gas flow pulses, detection of a sensor line occlusion is not possible.

Furthermore, it is also necessary in this case to know the pressure loss across the sensor line in order to correct the measurement at the second pressure sensor accordingly (see FIG. 2b).

In a simplified embodiment of the invention (not shown in the figures), the sensor line with the second gas pressure sensor connected thereto leads into the cavity via a second trocar, without the sensor line being connected to the first pressure and flow control unit. The second controllable valve is not required in this case. In this embodiment, an occlusion of the sensor line can occur only by comparing the measurement values between the first gas pressure sensor and the second gas pressure sensor. For this purpose, the first controllable valve in the supply line must be closed so that the measurement value of the first gas pressure sensor is not influenced by the gas inflow. When the first valve is closed, i.e. during an insufflation pause, both gas pressure sensors must display the same pressure, possibly taking into account the gas pressure losses across the lines (see above). If the pressure values deviate by more than 5 mmHg (in special cases more than 1 mmHg), an occlusion of the line or some other disturbance of the gas flow must be assumed.

### DESCRIPTION OF THE FIGURES

FIG. 1 shows the device according to the invention described at the outset. FIG. 1a shows the basic structure with the components of claim 1. Only the computing unit is not shown. The second controllable valve (11) enables a pulsatile gas flow into the cavity (8).

FIG. 1b shows the result of the pressure measurement of the second pressure sensor (p₁₂) compared to the actual pressure in the cavity (p₈) as a function of the gas flow in the sensor line. The upper curve shows the position of the second controllable valve (11), ‘on’ or ‘off’. The lower curves show the measured pressure at the second pressure sensor (12) or in the body cavity. The gas pulse in the sensor line results in a corresponding pressure pulse at the second pressure sensor (12) with approximately the same pulse width. In the case of an occlusion of the sensor line (right in the picture), there is a pressure increase at the second pressure sensor (12) that lasts significantly longer than the pulse width of the pressure pulse. The actual pressure in the body cavity (p₈) is constant in this example. If the measured pressure increase lasts significantly longer than the pulse width of the pressure pulse from the second controllable valve (11), without a corresponding pressure increase being registered by the first pressure sensor (5), it can be concluded that the sensor line (9) is occluded and an alarm can be triggered.

FIG. 1c shows the pressure profile in the event of an occlusion, i.e. when the sensor line (9) is removed from the second trocar without pulsing the second controllable valve. The pressure measured at the second pressure sensor remains constant in this case, even if the pressure p₈ in the cavity (8) increases.

FIG. 2a shows the embodiment of the invention with the check valve (15). FIG. 2b shows the result of the measurements: When the sensor line (9) is not connected to the second trocar (13), a short pressure pulse through the second controllable valve (11) leads to a longer-lasting pressure increase (p₁₂) at the second pressure sensor (12). As soon as the sensor line (9) is properly connected to the second trocar (13), the pressure (p₁₂) measured at the second pressure sensor (12) runs parallel to the actual pressure in the cavity (8). The pressure difference Δp shown in the chart results from the volume flow of the sensor line (9). With the valve (11) closed, the pressure difference would be smaller, but not equal to zero due to the pressure loss in the line (see Equation 1). This embodiment of the invention is also possible without the second controllable valve (11), which is therefore optional. However, if the second controllable valve (11) is not present, only the improper connection of the sensor line (9) to the second trocar (13) detectable, but not an occlusion between the cavity (8) and the second gas pressure sensor (12).

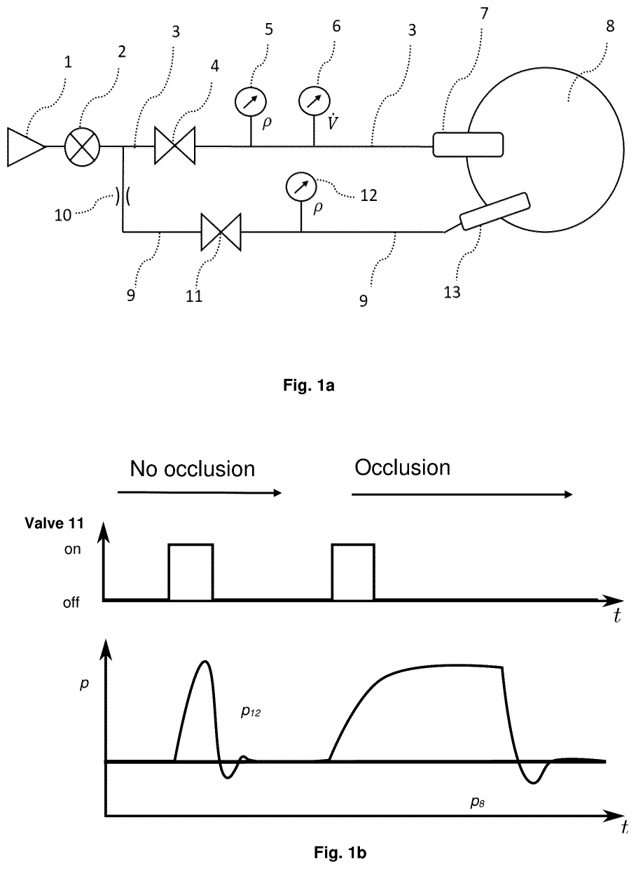

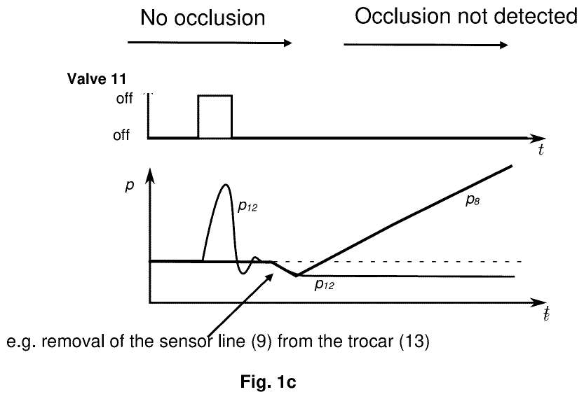

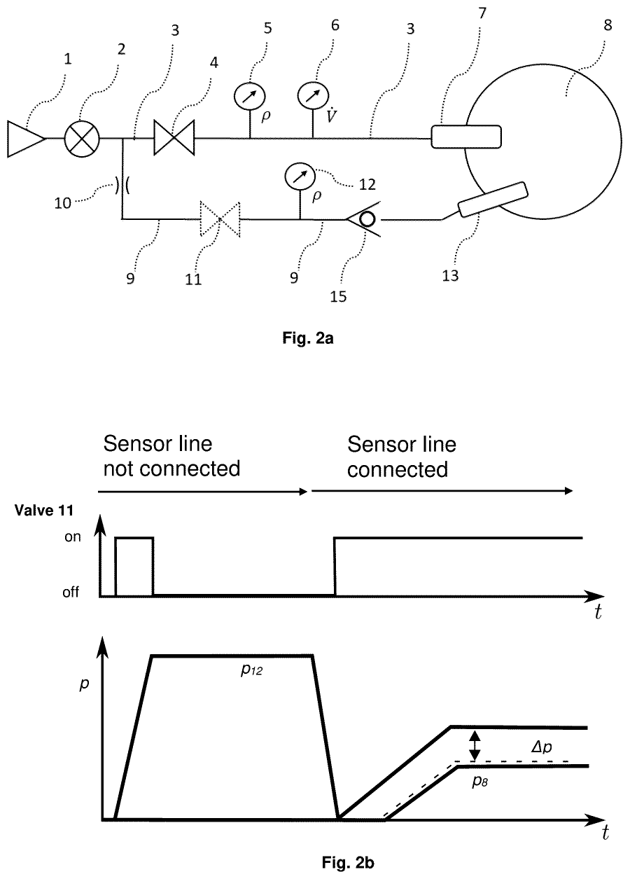

The reference numerals used in the figures have the following meanings:
(1) gas connection for a gas cylinder or domestic gas
(2) first pressure and flow control unit equipped with a proportional valve
(3) supply line
(4) first controllable valve,
(5) first gas pressure sensor
(6) first gas flow sensor
(7) first trocar
(8) cavity
(9) sensor line
(10) throttle (optional)
(11) second controllable valve
(12) second gas pressure sensor
(13) second trocar
(14) computing unit
(15) check valve

1. An insufflator for minimally invasive surgery, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second controllable valve (11) and a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
wherein the sensor line (9) is connected to the first pressure and flow control unit (2), and wherein the second controllable valve (11) allows a pulsatile gas flow controlled by the computing unit (14) into the cavity (8).

2. The insufflator for minimally invasive surgery of claim 1, further comprising a check valve (15), which opens already at a low back pressure of 8 to 12 mmHg on the side of the cavity and which is positioned in the sensor line between the second trocar (13) and the second gas pressure sensor (12).

3. An insufflator for minimally invasive surgery, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm configured for a gas flow of less than 1 L/min at 2-3 bar and with an optional throttle (10), with a second controllable valve (11) and a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12).

4. An insufflator for minimally invasive surgery, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
wherein the computing unit (14) is configured to correct the pressure losses of the supply line (3) with the first trocar (7) and of the sensor line (9) with the second trocar (13) and to compare the corrected measurement values of the first pressure sensor (5) and the second pressure sensor (6).

5. A method of detecting an occlusion of the sensor line during operation of an insufflator of claim 1, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second controllable valve (11) and a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
wherein the sensor line (9) is connected to the first pressure and flow control unit (2), and wherein the second controllable valve (11) generates a constant gas flow into the cavity (8),
wherein the measured pressure profile of the second gas pressure sensor (12) is monitored by the computing unit (14), a correction of the measurement data is performed on the basis of the pressure loss in the sensor line, and
wherein an alarm is triggered within 1 to 2 seconds in the event of a significant pressure increase of at least 2 mmHg.

6. A method for detecting an occlusion of the sensor line during operation of an insufflator of claim 1, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second controllable valve (11) and a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
wherein the sensor line (9) is connected to the first pressure and flow control unit (2), and wherein the second controllable valve (11) generates a pulsatile gas flow into the cavity (8),
wherein the measured pressure profile of the second gas pressure sensor (12) is monitored by the computing unit (14), and wherein an alarm is triggered in the event of a pressure increase lasting for more than twice the pulse width.

7. A method of detecting an occlusion of the sensor line during operation of an insufflator of claim 2, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second controllable valve (11) and a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
f) a check valve (15), which opens at a back pressure of 8 to 12 mmHg on the side of the cavity and which is positioned in the sensor line between the second trocar (13) and the second gas pressure sensor (12)
wherein the sensor line (9) is connected to the first pressure and flow control unit (2), and wherein the second controllable valve (11) generates a constant gas flow into the cavity (8),
wherein the measured pressure profile of the second gas pressure sensor (12) is monitored by the computing unit (14), a correction of the measurement data is performed on the basis of the pressure loss in the sensor line, and
wherein an alarm is triggered within 1 to 2 seconds in the event of a significant pressure increase of at least 2 mmHg.

8. A method of detecting an occlusion of the sensor line during operation of an insufflator of claim 4, comprising:
a) a gas connection for a gas cylinder or domestic gas (1)
b) a first pressure and flow control unit (2) equipped with a proportional valve,
c) a supply line (3) with a cross-section of 5.5 to 15 mm, configured for a gas flow of 0-50 L/min at 2-3 bar, having a first controllable valve (4), a first gas pressure sensor (5), a first gas flow sensor (6) and a connection to a first trocar (7) into a cavity (8),
d) a sensor line (9) with a cross-section of 2 to 5 mm having an optional throttle (10), configured for a gas flow of less than 1 L/min at 2-3 bar, having a second gas pressure sensor (12) and a connection to a second trocar (13) into the cavity (8),
e) a computing unit (14) for controlling the insufflation and evaluating the measurement data of the first gas pressure sensor (5) and the second gas pressure sensor (12),
wherein the computing unit (14) performs a correction of the pressure losses of the supply line (3) with the first trocar (7) and of the sensor line (9) with the second trocar (13),
performs a comparison of the corrected measurement values of the first pressure sensor (5) and the second pressure sensor (6),
and, if the corrected measurement values deviate from each other by more than 2 mmHg, triggers an alarm.
